(19) United States
(12) Patent Application Publication
(10) Pub. No.: US 2010/0267629 A1
(43) Pub. Date: Oct. 21, 2010

(54) Enterostatin as Therapeutic Agent for Hypoglycemia

(75) Inventors: David A. York, Logan, UT (US); MieJung Park, Logan, UT (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(73) Assignee: Board of Supervisors of Louisiana State University and Agricultural and Mechanical College, Baton Rouge, LA (US)

(21) Appl. No.: 12/280,015

(22) PCT Filed: Feb. 27, 2007
(86) PCT No.: PCT/US2007/062851

§ 371 Date: Oct. 17, 2008

Related U.S. Application Data
(60) Provisional application No. 60/778,082, filed on Feb. 28, 2006.

(51) Int. Cl. A61K 38/26; A61K 31/4025; A61K 31/70; A61K 31/549; A61P 3/10

(52) U.S. Cl. 514/7.2; 514/423; 514/23; 514/223.5

(57) ABSTRACT

It has been discovered that enterostatin injections into mice caused an increase in blood glucose levels within 15 minutes of injection, and the glucose levels remained high for up to an hour after injection. In addition, mice injected with enterostatin showed less of an initial decrease in blood glucose following an insulin injection. Enterostatin was also shown to decrease AMPK activity in both mice and human tissues, which is additional support that glucose production is increased after enterostatin injection. This ability to enhance glucose production indicates that enterostatin could be used to treat hypoglycemia.

The benefit of the filing dates of provisional application 60/778,082 filed 2 Feb. 2006 is claimed under 35 U.S.C. §119(e) in the United States, and is claimed under applicable treaties and conventions in all countries.

The development of this invention was partially funded by the Government under a grant from the National Institutes of Health (NIDDK), grant no. DK45278. The Government has certain rights in this invention.

### TECHNICAL FIELD

This invention pertains to a method to ameliorate or prevent hypoglycemia by administering a therapeutically effective amount of enterostatin.

### BACKGROUND ART

### Hypoglycemia

Hypoglycemia is a condition of abnormally low levels of sugar (glucose) in the blood. Normally, the levels of blood glucose are maintained within the range of about 70 to 110 mg/dL of blood. In hypoglycemia, the glucose levels fall below this range. Low levels of blood glucose can affect the function of many organ systems, especially the brain is very sensitive to low glucose levels. Hypoglycemia is uncommon in adults or in children older than 10 years old except as a side effect of diabetes treatment (e.g., too much insulin injected). However, hypoglycemia can result from other medications (e.g., sulfonylureas, pentamidine), diseases (viral hepatitis, cirrhosis of liver), hormone deficiencies (e.g., glucagons), enzyme deficiencies, kidney failure, and tumors (e.g., liver or pancreas).

Two types of hypoglycemia can occur in people who do not have diabetes: reactive (postprandial or after meals) and fasting (postabsorptive). Reactive hypoglycemia is not usually related to any underlying disease; while fasting hypoglycemia often is. In reactive hypoglycemia, symptoms usually appear within 6 hours after a meal is eaten.

The causes of most cases of reactive hypoglycemia are still open to debate. Suggestions include an increased sensitivity to the hormone epinephrine, or a decreased secretion of glucagon from the pancreas. A few uncommon causes of reactive hypoglycemia are known. Gastric (stomach) surgery, for instance, can cause hypoglycemia because of the rapid passage of food in the small intestine. Also, rare enzyme deficiencies which would be diagnosed early in life, such as hereditary fructose intolerance, may cause reactive hypoglycemia.

Fasting hypoglycemia is diagnosed when the blood glucose level is less than 50 mg/dL of blood some six hours or more after a meal, or in other situations in which glycogen has been depleted. Examples of causes of fasting hypoglycemia include certain medications, alcohol, hormonal deficiencies, some kinds of tumors, hepatic disease, metabolic disorders related to glycogen, and fructose metabolism.

Medications, including some used to treat diabetes, are the most common cause of hypoglycemia. Some non-diabetic medications include salicylates (e.g., aspirin taken in large doses), sulfa medicines (used to treat infections), pentamidine (used to treat pneumonia), and quinine (used to treat malaria). Some illnesses that affect the liver, pancreas, heart, or kidneys can cause hypoglycemia. Sepsis (overwhelming infection) is another cause of hypoglycemia. Alcohol consumption can also cause hypoglycemia.

Hormonal deficiencies (e.g., cortisol, growth hormone, glucagons, or epinephrine) may cause hypoglycemia in very young children, but not usually in adults. Some tumors (e.g., insulinomas) can cause hypoglycemia. Overproduction of insulin (hyperinsulinism), common in infants of diabetic mothers, can result in transient neonatal hypoglycemia. Enzyme deficiencies that affect the normal carbohydrate metabolism (e.g., fructose, galactose, glycogen, or other metabolites) can lead to persistent hypoglycemia.

Currently acute hypoglycemia is treated usually by giving a rapidly absorbed form of glucose (glucose tablets, candy, fruit juice) or by injection with glucagon. For persistent hypoglycemia, removal of the underlying cause is often the best treatment. The main drugs available to increase blood glucose currently are glucagon (for severe hypoglycemia) and diazoxide (Proglycem). Glucagon stimulates the liver to release large amounts of glucose and acts within 5 to 15 mins to restore blood sugar. Diazoxide increases blood sugar by inhibiting pancreatic insulin release and usually acts within 1 hr for a duration of about 8 hr.

Enterostatin

Enterostatin is the aminoterminal pentapeptide of procolipase that is released by proteolytic activity when procolipase is converted into colipase (9). The procolipase gene is expressed in the exocrine pancreas and the gastric and duodenal mucosa (25, 34, 53). In the gastric mucosa, the gene appears to be concentrated in enterochromaffin cells. More recently, procolipase and enterostatin were shown to be present in specific brain regions including the amygdala and hypothalamus (12).

Enterostatin Effects on Feeding Behavior. The peptide enterostatin has a dose-dependent and selective effect to inhibit fat intake in a number of dietary paradigms. The first criteria for establishing the physiological role of a peptide on feeding behavior is that which inhibits food or macronutrient intake in rats adapted to a three-choice macronutrient diet of fat, carbohydrate and protein (7, 36, 37). Enterostatin reduced intake of the fat macronutrient, but had no effect on either carbohydrate or protein intake. In a two-choice high-fat (HF) and low-fat (LF) diet paradigm experiment, enterostatin-reduced only intake of the HF diet, but not of the LF diet (15) Similarly, enterostatin reduced intake of single dietary source when the source was HF (17), but not when LF. The ability of enterostatin to selectively inhibit fat intake on a two- or three-choice feeding paradigm has been demonstrated after administration of enterostatin by either intraperitoneal, intracerebroventricular (icv), intraduodenal/intragastric, and near celiac arterial injection (15, 16, 19, 22, 27, 29, 52, 57). Similar to other gut peptides, enterostatin appeared to have at least two sites of action, one in the gastrointestinal tract and one in the central nervous system (20, 49, 57).

While the majority of the feeding studies with enterostatin have been performed in overnight fasted rats that have been previously adapted to the experimental diets, the selective effects towards dietary fat have been shown in free-feeding rats injected at the start of the dark cycle. The potency of enterostatin is reflected in the long duration of action on feeding, lasting up to six hours after a single injection in rats adapted to a six-hour feeding schedule, and lasting up to 24 hours after a single injection in rats adapted to ad-libitum feeding. Chronic icy administration of enterostatin from mini-osmotic pumps also attenuated the daily intake of dietary fat in rats fed either a single-choice HF diet or a two-choice HF/LF diet (15, 35). The decrease in daily food intake was accompanied by a reduction in fat deposition and body weight gain. However, in rats chronically treated with enterostatin and fed a low-fat diet for seven days, no significant reduction was seen in either energy intake or change in body weight gain. An intriguing characteristic of the response to enterostatin in both acute and chronic studies was that the reduction in intake of dietary fat is not compensated by an increase in the intake of other macronutrients when a dietary choice is available. This may result from a concomitant increase in corticotropin releasing hormone (CRH) secretion since enterostatin is known to activate the hypothalamic-pituitary-adrenal (HPA) axis (35).

Enterostatin has also been shown to reduce food intake in rabbits, sheep, and baboons (8, 30, 51). However, all of these studies were performed with single-choice diets. In humans, enterostatin administered by intravenous injection was found to reduce the subjective feeling of hunger (44), although has not been found to reduce food intake (43).

Enterostatin effects on fat intake appear to be expressed at both gastrointestinal and central nervous system (CNS) sites. The response to peripherally-administered enterostatin was found to be mediated through the hepatic vagus nerve; the response was abolished by either selective hepatic vagotomy or capsaicin treatment (32, 49). Within the CNS, enterostatin was found to act on both the amygdala and paraventricular nucleus (PVN) (12, 14, 20). Enterostatin inhibited fat intake by way of a pathway that contained both serotonergic (55) and kappa-opioidergic (38) neurons. Kappa-opioidergic agonists inhibited the enterostatin effects on feeding, and a K-opioidergic antagonist or nor-Binaltorphamine (nor-BNI) mimicked the effect of enterostatin on selective fat intake (1, 38). In contrast, the general serotonergic antagonist, metergoline but not a 5HT2 receptor antagonist, blocked the response to icy-administered enterostatin (57), and serotonin injections into the PVN inhibited dietary fat intake (10, 45).

A physiological regulator of feeding behavior must be effective at dose levels that are present in the animal. The in vivo concentration of enterostatin has not been established, due to problems in measuring enterostatin. Antibodies that are selective to enterostatin that could be used to analyze tissue levels of enterostatin have been difficult to find. The current values for enterostatin all appear very high, for example, plasma serum enterostatin of 5-40 nM in humans (4) and rats, cerebral spinal fluid enterostatin of 18-92 ng/ml, and brain enterostatin levels of 2.5 nmoles/g tissue (11). A suggestion of the existence of multiple forms of enterostatin in rats and in humans because of genetic polymorphisms in the enterostatin region of the procolipase parent molecule further complicates the efforts to measure enterostatin (11, 46). However, other data has disputed the suggestion of multiple forms (53, 54). Despite these measurement problems, enterostatin-like immunoreactivity has been shown to increase both in human serum and urine after a meal in a biphasic manner (4), and in lymph fluid of cats (50) and serum of rats after feeding (9).

Enterostatin regulation of insulin secretion. Several studies have shown that enterostatin inhibits insulin secretion (24, 26, 28, 39, 42, 47). In vivo perfusion of isolated islets and of the rat pancreas has been used to demonstrate that enterostatin directly inhibits insulin release from islet cells induced by either glucose, tolbutamide, or arginine. (39) Enterostatin (10⁻⁹ to 10⁻⁵ M) inhibited insulin secretion from islets incubated in the presence of 16.7 mM glucose in a dose-dependent manner. Enterostatin also inhibited insulin secretion stimulated by glybenclamide (5.0 and 10 μM), phorbol 12-myristate-13-acetate (TPA) (50 and 100 nM), and the kappa-opioid agonist U50,488 (100 nM). The inhibitory effect of enterostatin on TPA-induced insulin secretion was attenuated, but still remained in the absence of extracellular Ca²⁺. The enterostatin inhibition of insulin secretion was blocked by 8-Br-cAMP (1 mM), independent of extracellular Ca²⁺. Enterostatin reduced the increase in intracellular cyclic AMP content produced by U50,488 (100 nM), in a manner parallel with changes in insulin release (42).

In vivo studies also have shown a reduction in insulin levels without any changes in plasma glucose suggesting an improvement in insulin sensitivity (15, 35). This occurred after both peripheral and central administration of enterostatin, reflecting both direct effects on the islet cells and indirect effects by way of a reduction in vagal stimulation to the pancreas.

Other Effects of Enterostatin. Enterostatin also been shown to affect gastrointestinal motility and gastric emptying (21, 40). The inhibition of gastric emptying was observed only after intracerebroventricular administration of enterostatin, but not after either intraperitoneal or intragastric administration, suggesting that enterostatin also affects efferent vagal activity. However, the inhibitory effect of enterostatin on consumption of a high fat diet was not related to the slowdown of gastric emptying (21). Enterostatin also had direct effects on pig intestine to prolong the quiescent phase I period of peristalsis, which slows down the absorption of nutrients and prolongs intestinal transit time. Enterostatin may also reduce cholesterol levels (48).

Enterostatin also has shown a number of autonomic and endocrine effects in addition to the effect on insulin secretion. It enhanced corticosterone secretion (35) and sympathetic stimulation to brown adipose tissue (32, 33), which would increase thermogenesis (41). These responses, in addition to the suppression of dietary fat intake, help explain the reduction in weight gain and body fat that was seen in rats treated chronically with either peripheral or central enterostatin (15, 35).

Circulating enterostatin. Enterostatin absorption across the intestine was found to be limited and slow, occurring mainly into lymphatic system. Detailed information of the changes in plasma enterostatin or brain uptake of enterostatin after a meal currently exist that would allow a temporal comparison with the termination of feeding and the development of satiety. The data that are available indicate the rise in plasma immunoreactive-like enterostatin activity is slow and does not peak until at least 60 minutes after feeding, which is inconsistent with a theory that an increase in circulating enterostatin plays a role in the termination of the immediate meal.

The presence of procolipase mRNA in the CNS together with enterostatin-like immunoreactivity has been demonstrated (12). Enterostatin also was found at high levels in the cerebrospinal fluid of rats. A hypothesis that this central system is important in determining the appetite for dietary fat is consistent with the evidence that endogenous production of enterostatin is reciprocally related to voluntary selection of fat across and within rat strains.

Enterostatin Receptors. Based on the areas responding to enterostatin, receptors would be expected to be located in brain, pancreas, and the gastrointestinal tract. Enterostatin has been shown not to bind to the galanin or Neuropeptide Y1 receptors (17), kappa-opioid receptors or cholecystokinin A receptors (13) Low affinity enterostatin binding was shown to a brain membrane preparation (Kd 230 nM) (56) and to SK-N-MC neuroepithelioma cells (Kd 40 nM) (2). The dose-response curve to enterostatin is biphasic, exhibiting an inhibition of food intake at lower doses, but stimulation of food intake at higher doses (22). However, since enterostatin has been shown to be biologically active on food intake at extremely low doses compared to other peptides and to inhibit insulin secretion from isolated pancreatic islets at doses of 10⁻¹⁰ to 10⁻⁶M, a proposed low affinity casomorphin binding site probably is not the biologically important enterostatin receptor that inhibits fat intake and insulin secretion.

Affinity chromatography identified the beta subunit of ATP synthase as a putative receptor for enterostatin (2). Subsequent work with enterostatin analogs on the binding of iodinated-beta casomorphin (an antagonist of enterostatin) to purified protein (See Table 1) supported this suggestion (60). The receptor protein has also been shown to be localized on the plasma membrane of multiple tissues and to have a Kd of 2.5 nM on liver plasma membranes.

More recently, through indirect calorimetry, enterostatin has been shown to enhance fat oxidation in vivo, and that part of this effect is due to a direct action on muscle to increase fatty acid oxidation through a stimulation of the AMPkinase pathway. (59).

### DISCLOSURE OF INVENTION

We have shown that enterostatin injections into mice caused an increase in blood glucose levels within 15 minutes of injection, and the glucose levels remained high for up to an hour after injection. In addition, mice injected with enterostatin showed less of an initial decrease in blood glucose following an insulin injection. Enterostatin was also shown to decrease AMPK activity in both mice and human liver tissue, which is additional support that glucose production is increased after enterostatin injection. This ability to enhance glucose production indicates that enterostatin could be used to treat hypoglycemia.

### BRIEF DESCRIPTION OF THE DRAWINGS

FIG. 1 illustrates the effect over time of the injection of a control (saline) or of two different concentrations of enterostatin (25 μg and 5 μg/mouse) on the response of serum glucose to insulin injection in C57B1/6 male mice fasted for four hours prior to the simultaneous injection of insulin and enterostatin.

FIG. 2A illustrates the acute effect of an injection of enterostatin (5 μg/mouse) given 20 minutes prior to a glucose tolerance test (1.0 mg/gm body weight glucose injected intraperitoneally) in C57B1/6 male mice fasted for 18 hours.

FIG. 2B illustrates the acute effect of an injection of enterostatin (25 μg/mouse) given 20 minutes prior to a glucose tolerance test (1.0 mg/gm body weight glucose injected intraperitoneally) in C57B1/6 male mice fasted for 18 hours.

FIG. 3A illustrates the effect over time of an injection of enterostatin (25 μg/mouse) or of saline on the level of blood glucose, measured as changes in blood glucose from time zero (Delta Blood Glucose), in C57B1/6 male mice fasted for 18 hours prior to the injection.

FIG. 3B illustrates the effect overtime of an injection of enterostatin (25 μg/mouse) or of saline on the level of blood glucose, measured as a percentage change in blood glucose, in C57B1/6 male mice fasted for 18 hours prior to the injection.

FIG. 4 illustrates the results of a Western Blot Analysis assaying for in vivo AMPK activity in two tissues (liver and hypothalamus) from mice that were fasted 18 hours prior to injection with saline or enterostatin (5 or 25 μg/mouse) and then sacrificed at either 30 or 60 min after injection.

FIG. 5A illustrates the effects of various concentrations of enterostatin on pAMPK activity after 15 min incubation both with and without an antibody to the enterostatin receptor (anti F1-ATPase beta subunit), as shown in a Western blot analysis in human liver cells (HepG2) in the presence of 5 mM glucose.

FIG. 5B illustrates the effects of various concentrations of β-casomophin on pAMPK activity after 15 min incubation both with and without an antibody to the enterostatin receptor (anti F1-ATPase beta subunit), as shown in a Western blot analysis in human liver cells (HepG2) in the presence of 5 mM glucose.

FIG. 6 illustrates the effect of enterostatin on the β-casomophin stimulation of pAMPK activity after 15 min incubation, as shown in a Western blot analysis in human liver cells (HepG2) in the presence of 5 mM glucose.

FIG. 7 illustrates the effects of enterostatin on pAMPK and PKArIIβ activity after 1 hr incubation as shown in a Western blot analysis in human liver cells (HepG2) in the presence of 5 mM glucose.

### MODES FOR CARRYING OUT THE INVENTION

### Example 1

Materials and Methods.

Animals: C57B1/6 male mice were purchased from The Jackson Laboratory (Bar Harbor, Me.) at 6 weeks of age. The mice were initially housed in groups of three in acrylic cages in a room with a 12-hour light/dark cycle and with controlled temperature (22 to 23° C.) and with free access to water. The mice were fed a high fat diet (4.78 kcal/g, 56% energy as fat; Research Diets Inc, Brunswick, N.J.). The composition of the diet has been previously described (15). Body weights were measured three time per week. At 8 weeks of age, the mice were switched to single housing.

Peptide and antibodies: Enterostatin was synthesized by solid-phase chemistry purified by high performance liquid chromatography, and estimated to be greater than 90% purity by the Core Laboratory of Louisiana State University Medical Center (New Orleans, La.). Antibodies against phosphor-AMP kinase (pAMPK) and AMP kinase (AMPK) were purchased from Upstate Biotechnology (Lake Placid, N.Y.).

Peptide Injections: Enterostatin was dissolved in 0.1 ml saline (0.9% NaCl w/v), and given as a single dose of either 5 μg or 25 μg/mouse. Either enterostatin or saline (control) was injected intraperitoneally. In the experiments involving insulin, insulin (0.75 mU/g body weight) or saline (0.1 ml/10 g body weight; control) was injected intraperitoneally.

### Example 2

### Effect of Enterostatin on Serum Blood Glucose

Groups of mice (n=4 to 7) were subjected to glucose and insulin tolerance tests and to a study of the response of blood glucose to intraperitoneal injection of enterostatin. There was a minimum of 1 week between each test for the mice to recover.

Enterostatin Effect on Blood Glucose Response to Insulin. To test the effect of enterostatin on the blood glucose response to exogenous insulin, mice were fasted for 4 hours, and insulin (0.75 mU/gm body weight) or saline (0.1 ml/10 gm body weight) was injected intraperitoneally at time zero. Enterostatin (5 or 25 ug/mouse in 0.1 ml saline vehicle) or saline vehicle was also injected intraperitoneally (ip) at time zero. Blood samples were taken from the tail vein immediately before the injections, and then at 15, 30, 45 and 60 minutes afterwards. The samples were assayed for glucose on a glucometer (Ascencia Elite XL, Bayer, Pittsburgh, Pa.).

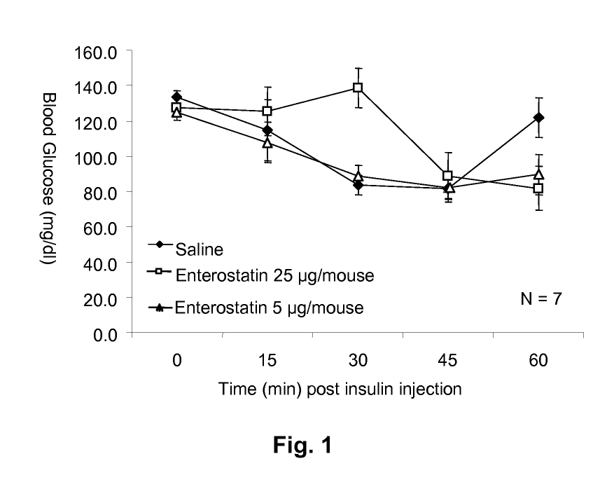

The effect of enterostatin on the blood glucose response to insulin is shown in FIG. 1. Injection of insulin reduced the blood glucose as expected in the control mice with the blood glucose level reaching a minimum of 82 mg/100 ml at 30 min. The glucose level then returned to a level above zero time levels. Mice pretreated with the lower dose of enterostatin (5 μg/mouse) showed a similar reduction in glucose in the first 30 min, but at 60 min, the glucose level had not returned to zero-time levels. At the higher dose of enterostatin, the initial fall in blood glucose in response to insulin was delayed for 30 minutes. However, once the glucose decreased at 45 min, the level remained below the zero-time level even after 60 min. This indicates after an injection of enterostatin, blood glucose is increased for at least the first 30 min.

Enterostatin Effect on Glucose Tolerance Tests: For glucose tolerance tests, mice were fasted overnight (18 hours). Enterostatin (5 or 25 ug/mouse in 0.1 ml saline vehicle) or saline vehicle was injected intraperitoneally (ip) 20 minutes before an intraperitoneal injection of glucose (1.0 mg/gm body weight). Blood samples were taken from the tail vein immediately before glucose administration, and then at 15, 30, 45 and 60 min after glucose injection. The blood samples were assayed for glucose using a glucometer.

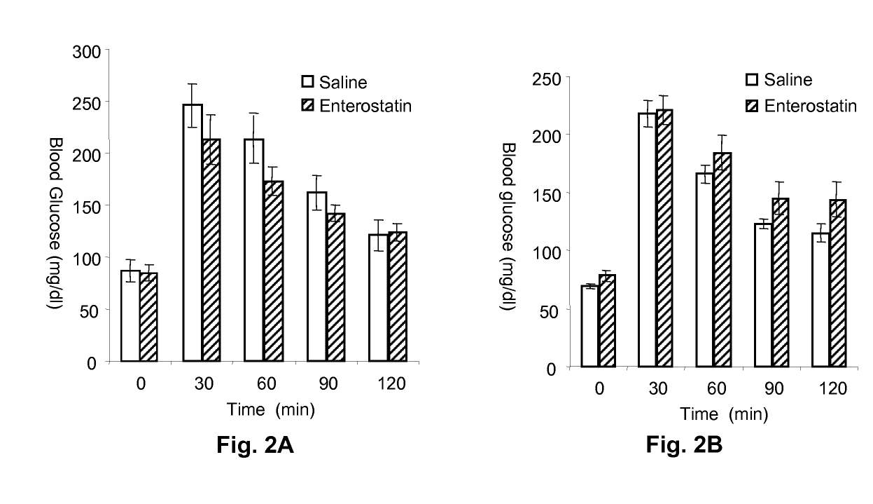

The effect of enterostatin in the glucose tolerance tests is shown in FIGS. 2A and 2B. There were no differences in the clearance of blood glucose in control and enterostatin-treated mice at either dose (5 μg/mouse (FIG. 2A) and 25 μg/mouse (FIG. 2B)). Since previous reports had suggested that enterostatin increased insulin sensitivity, the results in FIGS. 1, 2A and 2B were surprising. These results suggest that enterostatin may initially increase glucose production.

Enterostatin Effect on Fasting Blood Glucose. To directly test the effect of enterostatin on blood glucose, mice were fasted for 18 hours, and enterostatin (25 μg/mouse) or 0.1 ml saline vehicle was injected intraperitoneally at zero time. Blood samples were taken from the tail vein at 15, 30, 45 and 60 min after enterostatin injection. The blood samples were assayed for glucose using a glucometer.

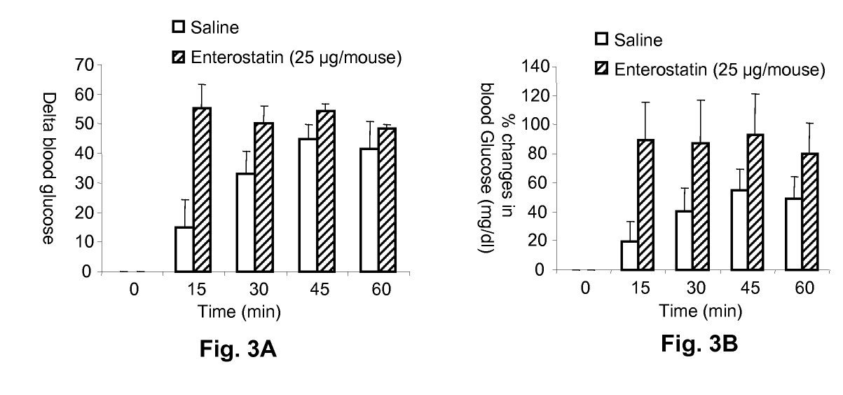

The intraperitoneal injection of enterostatin caused a rapid increase in blood glucose levels compared to the saline control group, as shown in FIGS. 3A and 3B. Although mice injected with saline vehicle also showed a rise in glucose over the 60 min time course, the increase in enterostatin-treated mice was significantly greater at both 15 and 30 min. These data suggest that enterostatin enhances hepatic gluconeogenesis at least in the first 15 min of injection.

### Example 3

### Enterostatin Effects on AMPK Activity In Vivo

A separate set of mice (n=6/group) were fasted overnight (18 hours) before injection with either saline vehicle (0.1 ml) or enterostatin (5 or 25 ug/mouse) intraperitoneally. The mice were sacrificed by cervical dislocation either at 30 min (certain enterostatin-treated groups) or 60 min (both certain enterostatin- and vehicle-treated groups) after the injection. The tissues of liver and hypothalamus were rapidly dissected and frozen in liquid nitrogen. The tissues were stored at −80 C before processing for AMPK activity. For AMPK activity, the tissues were unfrozen, the cells lysed, and the cytosolic proteins subjected to Western blot analysis for AMPK and pAMPK activity.

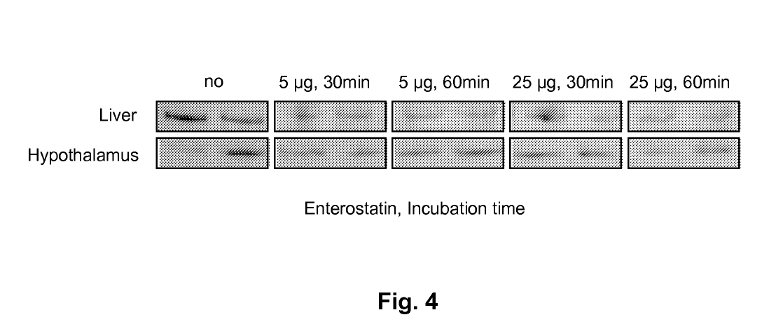

FIG. 4 illustrates the results of the Western blot analysis for pAMPK activity in liver and hypothalamus tissues. As shown in FIG. 4, liver pAMPK levels were reduced at both 30 and 60 min after injection of enterostatin at even the lower dose. Total AMPK was unaffected by the treatments. Hypothalamic pAMPK was unaltered as enterostatin may not cross the blood-brain barrier. These data are consistent with the known effects of muscle AMPK to regulate fatty acid oxidation and hepatic AMPK to inhibit gluconeogenesis. These data suggest that enterostatin may enhance hepatic gluconeogenesis through an inhibition of pAMPK.

### Example 4

### In Vitro Enterostatin Effects on AMPK Activity in Human Liver Cells

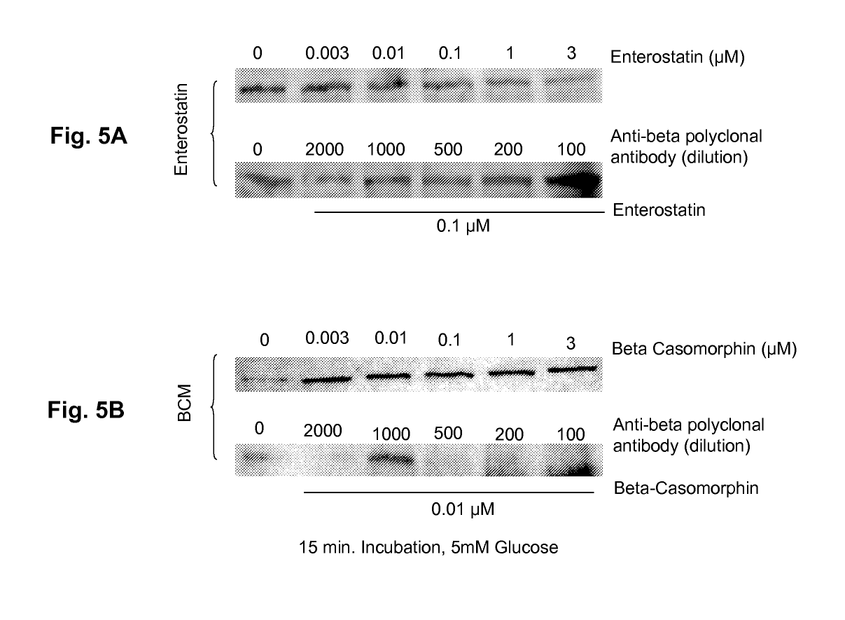

Human liver cells (HepG2 cell line, American Type Culture Collection, Manassas, Va.) were grown and maintained in Dulbecco's modified eagle's medium (Gibco, Carlsbad, Calif.) containing 10% fetal bovine serum, penicillin (100 I.U./ml), and streptomycin (100 μg/ml). The cells were then incubated for 15 min with 5 mM glucose and various concentrations of enterostatin (0.003, 0.01, 0.1, 1, and 3 μM) and/or of its antagonist β-casomorphin (BCM) (0.003, 0.01, 0.1, 1, and 3 μM). After incubation, the cells were lysed and the cytosolic proteins subjected to Western blot analysis for AMPK and pAMPK. The results for pAMPK activity are shown in FIG. 5A (enterostatin) and FIG. 5B (BCM). Higher doses of enterostatin inhibited AMPkinase as shown by the reduction in pAMPK (FIG. 5A). This effect was blocked by the presence of an antibody to the enterostatin receptor (anti F1-ATPase beta subunit). In contrast, β-casmorphin activated AMPkinase as shown by the increase in pAMPK levels even at the lowest dose used (FIG. 5B). Once again, incubation in the presence of a receptor antibody (anti F1-ATPase beta subunit) blocked this effect.

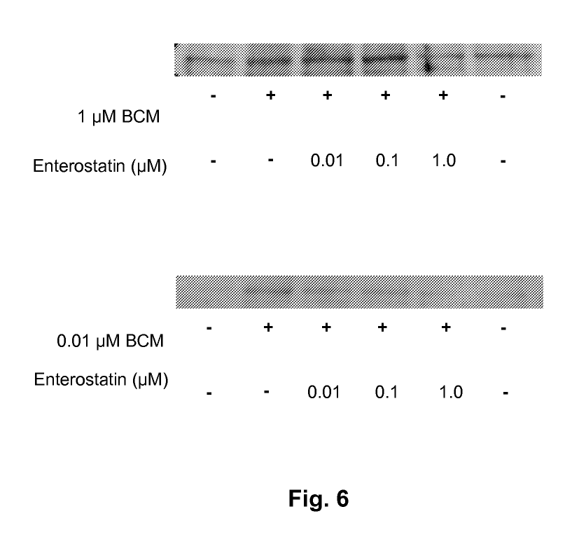

Incubation of HepG2 cells with β-casomorphin (0.1 or 1.0 μM) increased pAMPK levels (FIG. 6). The stimulation of pAMPK levels by β-casomorphin was reversed dose-dependently when the cells were also incubated in the presence of increasing concentration (0.01, 0.1, and 1.0 uM) of enterostatin, as shown in FIG. 6.

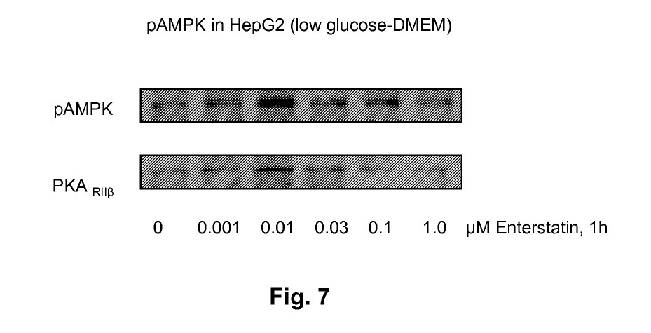

In a second experiment, human liver cells (HepG2 cell line, American Type Culture Collection, Manassas, Va.) were grown and maintained in Dulbecco's modified eagle's medium (Gibco, Carlsbad, Calif.) containing 10% fetal bovine serum, penicillin (100 I.U./ml), and streptomycin (100 μg/ml). The cells were then incubated for 60 min with 5 mM glucose and various concentrations of enterostatin (0.001, 0.01, 0.03, 0.1, 1.0 μM). After incubation, the cells were lysed and the cytosolic proteins subjected to Western blot analysis for pAMPK and PKARIIβ. The results are shown in FIG. 7.

Incubation of HepG2 cells with enterostatin had dose-dependent effects on pAMPK and PKARIIβ. After one hour incubation, rather than the 15 min incubation in FIGS. 5A, 5B, and 6, an increase in pAMPK levels was seen with a peak effect at the 0.01 μM dose. WE believe this is a reciprocal response to the earlier enterostatin inhibition of AMP kinase activity which would increase glucose production in the HepG2 cells. In addition the increase in PKARIIβ, again peaking at a dose of 10 nM enterostatin, is consistent with a theory that enterostatin activates the adenyl cyclase pathway which would increase glycogen breakdown and promote glucose production.

Thus, we have shown that enterostatin regulates AMPK activity, and through this action can have direct effects on tissue metabolism. We have also shown that enterostatin would promote glucose production by the liver. This ability to enhance glucose production will have a therapeutic effect for the treatment of hypoglycemia.

Miscellaneous

The term “enterostatin” used herein and in the claims refers to the peptide enterostatin, its derivatives and analogs. The terms “derivatives” and “analogs” are understood to be compounds that are similar in structure to enterostatin and that exhibit a qualitatively similar effect to the unmodified enterostatin. Examples of such derivatives and analogs are well known and are described in Table 1.

TABLE 1
The effect of enterostatin analogs on the binding of ¹²⁵I-β-
casomorphin₁₋₇ to F₁-ATPase β-subunit and food intake

| Enterostatin | Binding of ¹²⁵I-β- | Intake of high | |
|---|---|---|---|
| Analogs | casomorphin₁₋₇ | fat diet^{a} | Reference |
| APGPR | Increased | Suppression | (Lin et al., 1994) |
| PDP | Increased | Suppression | (Lin et al., 1994) |
| APGPY | Increased | Suppression | (Park et al., 2004) |
| YPGPR | Increased | Suppression | (Park et al., 2004) |
| VPDPR | Increased | Suppression | (Lin et al., 1994) |
| APGPRCY | Increased | Suppression | (Lin et al., 1994) |
| VPDPRCY | Increased | Suppression | (Lin et al., 1994) |
| YPDPR | Increased | Suppression | (Lin et al., 1994) |
| YVPDPR | Increased | Suppression | (Lin et al., 1994) |
| YGGAPGPR | Increased | Suppression | (Berger et al., 2002) |
| | | | (Park et al., 2004) |
| CYDPGPR | Displaced | Stimulation | (Lin et al., 1994) |
| CYAPGPR | Displaced | Stimulation | (Lin et al., 1994) |
| YPFPGPI | Displaced | Stimulation | (Lin et al., 1994) |
| APGPRY | Increased | No changes | (Park et al., 2004) |
| PGP | Increased | No changes | (Park et al., 2004) |
| PGPCY | Increased | No changes | (Park et al., 2004) |
| HP | Increased | No changes^{b} | (Lin et al., 1994) |
| DPGPR | Displaced | No changes | (Lin et al., 1994) |
| VPDPR-NH2 | Displaced | No changes | (Lin et al., 1994) |

^{a}In overnight fasted rats.
^{b}Only inhibits food intake as cyclo-HP.

The term “enterostatin agonist” as used herein refers to a molecule that selectively increases serum glucose by binding to the F₁-ATPase β-subunit in the plasma membrane or an alternative enterostatin receptor in the plasma membrane. As used herein, enterostatin agonist can include mimetics of enterostatin. An enterostatin agonist can also act, for example, by increasing the binding ability of enterostatin, or by favorably altering the conformation of the enterostatin receptor.

The term “enterostatin antagonist” as used herein refers to a compound that selectively inhibits or decreases the translocation of the F₁-ATPase β-subunit into the plasma membrane in tissues in which enterostatin would increase the translocation. An antagonist can act by any antagonistic mechanism, such as by binding to enterostatin or to F₁-ATPase β-subunit or an alternative enterostatin receptor, thereby inhibiting binding between enterostatin and the F₁-ATPase β-subunit or the alternative enterostatin receptor. An enterostatin antagonist can also act indirectly, for example, by modifying or altering the native conformation of either enterostatin or F₁-ATPase β-subunit.

The term “therapeutically effective amount” as used herein refers to an amount of enterostatin or its agonists sufficient to increase serum glucose to a statistically significant degree (p<0.05). The term “therapeutically effective amount” therefore includes, for example, an amount sufficient to increase glucose production to maintain a normal level of serum glucose. The dosage ranges for the administration of enterostatin are those that produce the desired effect. Generally, the dosage will vary with the age, weight, condition, feeding history, sex of the patient, and medical history. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the level of serum glucose by methods well known to those in the field. Moreover, enterostatin can be applied in pharmaceutically acceptable carriers known in the art. The application can be oral, by injection, or topical.

The present invention provides a method of treating, or ameliorating hypoglycemia, comprising administering to a subject at risk for hypoglycemia, a therapeutically effective amount of enterostatin or its agonists. The term “ameliorate” refers to a decrease or lessening of the symptoms or signs of hypoglycemia.

### REFERENCES

1. Barton C., L. Lin, D. A. York and G. A. Bray. Differential effects of enterostatin, galanin and opioids on high-fat diet consumption. Brain Res. 702:55-60, 1995
2. Berger K, Sivars U, Winzell M S, Johansson P, Hellman U, Rippe C, and Erlanson-Albertsson C. Mitochondrial ATP synthase—a possible target protein in the regulation of energy metabolism in vitro and in vivo. Nutr Neurosci 2002; 5: 201-210.
3. Berger K, Winzell M S, Erlanson-Albertsson C. Binding of enterostatin to the human neuroepithelioma cell line SK-N-MC. Peptides 19: 1525-1531, 1998.
4. Bowyer, R. C., Rowston, W. M., Jehanli, A. M. T., Lacey, J. H., Hermon-Taylor, J., The effect of a satiating meal on the concentrations of procolipase activation peptide in the serum and urine of normal and morbidly obese individuals, Gut., 34, 1520-1525, 1993.
5. Chang S, Park S, Kim S and Kang C. Interaction of the C-terminal domain of p43 and the α-subunit of ATP synthase: its functional implication in endothelial cell proliferation. J. Biol. Chem. 277; 8388-8394, 2001.
6. Das B, Mondrogan M. O., Sadeghian M, Hatcher V B and Norin A J. A novel ligand in lymphocyte mediated cytotoxicity: expression of beta subunit of H+ transporting ATP synthase on the surface of tumor cell lines. J. Exp. Med. 180: 273-81, 1994
7. Erlanson-Albertsson, C., Jie, M., Okada, S., York, D., Bray, G. A., Pancreatic procolipase propeptide, enterostatin, specifically inhibits fat intake, Physiol. Behav., 49, 1191-1194, 1991.
8. Erlanson-Albertsson, C., Larsson, A., A possible physiological function of procolipase activation peptide in appetite regulation, Biochimie, 70, 1245-1250, 1988.
9. Erlanson-Albertsson, C., York, D., Enterostatin—A peptide regulating fat intake, Obesity Res, 5, 360-372, 1997.
10. Halford J, Smith B K, Blundell J. Serotonin (5HT) and serotonin receptors in the regulation of macronutrient intake In: Neural and Metabolic Control of Macronutrient intake eds H R Berthoud and R J Seeley, pp 425-446 CRC Press, Boca Raton. 2000.
11. Imamura, M., Sumar, N., Hermon-Taylor, J., Robertson, H. J. F., Prasad, C., Distribution and characterization of enterostatin-like immunoreactivity in human cerebrospinal fluid. Peptides, 19: 1385-91, 1998.
12. Lin L, Braymer H D and York D A. Procolipase gene and enterostatin expression in the rat brain. FASEB J; 2002; A783.
13. Lin L, S. R. Thomas, G. Kilroy, G. J. Schwartz and D. A. York. The enterostatin inhibition of dietary fat intake is dependent upon CCKA receptors. Am. J. Physiology August; 285(2):R321-8, 2003.
14. Lin L., D. A. York. Amygdala enterostatin induces c-Fos expression in regions of hypothalamus that innvervate the PVN. Brain Res. 2004 Sep. 10; 1020(1-2):147-53.
15. Lin, L., Chen, J., York, D. A., Chronic icy enterostatin preferentially reduced fat intake and lowered body weight, Peptides, 18, 657-661, 1997.
16. Lin, L., G. Bray, and D. A. York. Enterostatin suppresses food intake in rats after near celiac and intracarotid arterial injection. Am. J. Physiol. Reg Integr Comp Physiol. 2000 May; 278(5):R1346-51.
17. Lin, L., Gehlert, D. R., York, D. A., Bray, G. A., Effect of enterostatin on the feeding responses to galanin and NPY, Obes. Res., 1, 186-192, 1993.
18. Lin, L., Umahara, M. York, D. A., Bray, G. A., β-casomorphins stimulate and enterostatin inhibits the intake of dietary fat in rats. Peptides, 19, 325-331, 1998.
19. Lin, L., York, D. A., Enterostatin actions in the amygdala and PVN to suppress feeding in the rat, Peptides, 18, 1341-1347, 1997.
20. Lin, L., York, D. A., Changes in the microstructure of feeding after administration of enterostatin into the paraventricular nucleus and the amygdala, Peptides, 19, 557-562, 1998.
21. Lin, L., York, D. A., Comparisons of the effects of enterostatin on food intake and gastric emptying in rats, Brain Res, 745, 205-209, 1997.
22. Lin, L., Okada, S., York, D. A., Bray, G. A., Structural requirements for the biological activity of enterostatin, Peptides, 15, 849-854, 1994.
23. Martinez L O, Jacquet S, Esteve J P, Rolland C, Cabezon E, Champagne E, Pineau T, Georgeaud V, Walker J E, Terce F, Collet X, Perret B, and Barbaras R. Ectopic beta-chain of ATP synthase is an apolipoprotein A-I receptor in hepatic HDL endocytosis. Nature (Lond) 2003; 421: 75-79.
24. Mei J, Bourras M, Erlanson-Albertsson C. Inhibition of insulin by intraduodenally infused VPDPR in rats. Peptides 18: 651-657, 1997
25. Mei, J., Bowyer, R. C., Jehanli, A. M. T., Patel, G., Erlanson-Albertsson, C., Identification of enterostatin, the pancreatic procolipase activation peptide, in the intestine of rat: effect of CCK-8 and high-fat feeding, Pancreas, 8, 488-493, 1993.
26. Mei, J., Cheng, Y., Erlanson-Albertsson, C., Enterostatin—its ability to inhibit insulin secretion and to decrease high-fat food intake, Int. J. Obesity, 17, 701-704, 1993.
27. Mei, J., Erlanson-Albertsson, C., Effect of enterostatin given intravenously and intracerebroventricularly on high-fat feeding in rats, Regul. Pept., 41, 209-218, 1992
28. Mei, J., Erlanson-Albertsson, C., Plasma insulin response to enterostatin and effect of adrenalectomy in rat, Obes. Res., 4, 513-519, 1996.
29. Mei, J., Erlanson-Albertsson, C., Role of intraduodenally administered enterostatin in rat: inhibition of food intake, Obes. Res., 4, 161-165, 1996.
30. Miner, J. L., Erlanson-Albertsson, C., Paterson, J. A., Baile, C. A., Reduction of feed intake in sheep by enterostatin, the procolipase activation peptide, J. Anim. Sci., 72, 1578-1582, 1994.
31. Moser t ET AL Angiostatin binds ATP synthase on the surface of human endothelial cells. Proc. Natl. Acad. Sci. 96: 2811-2816, 1999
32. Nagase H, A. Nakajimia, H. Sekihara, D. A. York and G. A. Bray. Regulation of feeding behavior, gastric emptying, and sympathetic nerve activity to interscapular brown adipose tissue by galanin and enterostatin: the involvement of vagal-central nervous system interactions. J. Gastroenterol. November; 37 Suppl 14:118-27, 2002.
33. Nagase H., G. A. Bray and D. A. York. Effect of galanin and enterostatin on sympathetic nerve activity to interscapular brown adipose tissue. Brain Res. 709:44-50, 1996.
34. Okada S., D. A. York and G. A. Bray. Procolipase mRNA: Tissue localization and effects of diet and adrenalectomy. Biochem. J. 292:787-789, 1993
35. Okada, S., Lin, L., York, D. A., Bray, G. A., Chronic effects of intracerebral ventricular enterostatin in Osborne-Mendel rats fed a high-fat diet, Physiol. Behav., 54, 325-330, 1993.
36. Okada, S., York, D. A., Bray, G. A., Mei, J., Erlanson-Albertsson, C., Differential inhibition of fat intake in two strains of rat by the peptide enterostatin, Amer. J. Physiol., 262, R1111-R1116, 1992.
37. Okada, S., York, D. A., Bray, G. A., Erlanson-Albertsson, C., Enterostatin (Val-Pro-Asp-Pro-Arg) the activation peptide of procolipase selectively reduces fat intake, Physiol. Behav., 49, 1185-1189, 1991.
38. Ookuma, K. C., Barton, C., York, D. A., Bray, G. A., Effect of enterostatin and kappa-opioids on macronutrient selection and consumption, Peptides, 18, 785-791, 1997.
39. Ookuma, M. and D. A. York. Inhibition of insulin release by enterostatin. Int. J. Obesity 22:800-805, 1998.
40. Pierzynowski, S. G., Erlanson-Albertsson, C., Podgurniak, P., Kiela, P., Weström, B., Possible integration of the electrical activity of the duodenum and pancreas secretion through enterostatin, Biomed. Res., 15, 257-260, 1994.
41. Rippe, C, berger K, Boiers C, Ricquier D and Erlanson-Albertsson C. Effect of high fat diet, surrounding temperature and enterostatin on uncoupling protein gene expression. Am. J. Physiol. 279; E293-E300, 2000.
42. Rodriguez-Gallardo J, Silvestre R A, Marco J Inhibitory effect of enterostatin on the beta cell response to digestive insulinotropic peptides. Int J Obes Relat Metab Disord 23(8):787-92, 1999
43. Rössner, S., Barkeling, B., Erlanson-Albertsson, C., Larsson, P., Wahlin-Boll, E., Intravenous enterostatin does not affect single meal food intake in man, Appetite, 34, 37-42, 1995.
44. Smeets, M., P. Geiselman, G. A. Bray and D. A. York. The effect of oral enterostatin on hunger and food intake in human volunteers. FASEB J 13(5):A871, 1999 (Abs).
45. Smith B. K., D. A. York, and G. A. Bray. Chronic d-fenfluramine treatment reduces fat intake independent of macronutrient preference. Pharmacology Biochem. & Behavior. 60:105-114, 1998.
46. Sörhede, M., Rippe, C., Mulder, H., Erlanson-Albertsson, C., Enterostatin is produced in three different forms in the rat intestine, Int. J. Obesity, 19, 115, 1995.
47. Tadayyon M., S Liou, C P Briscoe, G Badman, D S Eggleston, J R S Arch, D A York. Structure-function studies on enterostatin inhibition of insulin release. Intl J Diabetes & Metabolism 10:14-21, 2002.
48. Takenaka Y, Nakamura F, Yamamoto T, and Yoshikawa M. Enterostatin (VPDPR) and its peptide fragment DPR reduce serum cholesterol levels after oral administration in mice. Biosci. Biotechnol. Biochem. 2003; 67: 1620-1622.
49. Tian, Q., Nagase, H., York, D. A., Bray, G. A., Vagal-central nervous system interactions modulate the feeding response to peripheral enterostatin, Obes. Res., 2, 527-534, 1994.
50. Townsley, M. I., Erlanson-Albertsson, C., Ohlsson, A., Rippe, C., Reed, R. K., Enterostatin efflux in cat intestinal lymph: Relation to lymph flow, hyaluronan and fat absorption, Am. J. Physiol., 271, G714-G721, 1996.
51. Weatherford, S. C., Lattemann, D. F., Sipols, A. J., Chavez, M., Kermani, Z. R., York, D. A., Bray, G. A., Porte, Jr., D. Woods, S. C., Intraventricular administration of enterostatin decreases food intake in baboons, Appetite, 19, 225, 1992.
52. White C L, Bray G A, and York D A. Intragastric beta-casomorphin(1-7) attenuates the suppression of fat intake by enterostatin. Peptides 2000; 21: 1377-1381.
53. Winzell, M. S., M. E. Lowe, and C. Erlanson-Albertsson. Rat gastric procolipase: sequence, expression, and secretion during high-fat feeding. Gastroenterology 115: 1179-1185, 1998.

54. Wu, Y J, D Hughes, L Lin, D H Braymer and D A York. Comparative study of enterostatin sequence in five rat strains and enterostatin bind proteins in rat and chicken serum. Peptides March; 23: 537-544, 2002.
55. York, D. A, Waggener, J., Bray, G. A., Brain amine responses to peripheral enterostatin, Int. J. Obes., 18, 102, 1994 (Abs.).
56. York, D. A. and Lin L. Enterostatin: A peptide regulator of fat ingestion. In: Pennington Symposium Series, Molecular Biology of Obesity, Vol. 4, pp. 281-297 (G. A. Bray and D. Ryan, eds.), Louisiana State University Press. Baton Rouge, USA, 1996.
57. York, D. A., L. Lin, B. Smith, J. Chen. Enterostatin as a regulator of fat intake. In: Neural and metabolic control of macronutrient selection. H. R. Berthoud and R. J. Seeley (eds.), Chap 20, pp 295-308, 2000. CRC Press (Boca Raton, USA).
58. York, D. A., L. Lin, B. Smith, J. Chen. Enterostatin as a regulator of fat intake. In: Neural and Metabolic Control of Macronutrient Intake, H. Berthoud, R. Seeley (eds.), Chap. 20, pp 295-308, Boca Raton, Fla.: CRC Press, 2000.
59. Lin, L., Park, M., Hulver, M., York, D. A., Different metabolic responses to central and peripheral injection of enterostatin, Am. J. Physiol. Regul. Integr. Comp. Physiol., 2005 Dec. 8 [Epub ahead of print].
60. Park, M., Lin, L., Braymer, T. S., Smith, P. M., Harrison, D. H., York, D. A., The F1-ATPase beta-subunit in the putative enterostatin receptor, Peptides, December; 25:2127-33, 2004.

The complete disclosures of all references cited in this specification are hereby incorporated by reference. In the event of an otherwise irreconcilable conflict, however, the present specification shall control.

We claim:

1. A method of ameliorating or treating the symptoms of hypoglycemia in a mammal in need of such treatment, comprising administering to the mammal a therapeutically effective amount of enterostatin or its analogs.

2. The method of claim 1, wherein the enterostatin is administered intraperitoneally.

3. The method of claim 1, additionally comprising administering to the mammal a compound selected from the group consisting of glucagon, glucose, and diazoxide.

4. The method of claim 1, wherein the hypoglycemia is a result of one or more causes selected from the group consisting of hyperinsulinism, gastric surgery, kidney failure, viral hepatitis, sepsis, cirrhosis of liver, alcoholism, hormone deficiency, enzyme deficiency, liver cancer, pancreatic cancer, medication with salicylates, medication with sulfa medicines, medication with pentamidine, and medication with quinine.

5. A method to increase the blood sugar level in a mammal, comprising administering to the mammal a therapeutically effective amount of enterostatin or its analogs.

6. The method of claim 5, wherein the enterostatin is administered intraperitoneally.

7. The method of claim 5, additionally comprising administering to the mammal a compound selected from the group consisting of glucagon, glucose, and diazoxide.

8. The method of claim 5, wherein the low blood sugar is a result of one or more causes selected from the group consisting of hyperinsulinism, gastric surgery, kidney failure, viral hepatitis, sepsis, cirrhosis of liver, alcoholism, hormone deficiency, enzyme deficiency, liver cancer, pancreatic cancer, medication with salicylates, medication with sulfa medicines, medication with pentamidine, and medication with quinine.
